(19) United States
(12) Patent Application Publication
(10) Pub. No.: US 2017/0095623 A1
(43) Pub. Date: Apr. 6, 2017

(54) Device for Vaporization of Phyto Material

(71) Applicant: Michael Trzecieski, Mid Levels (HK)

(72) Inventor: Michael Trzecieski, Mid Levels (HK)

(21) Appl. No.: 15/281,184

(22) Filed: Sep. 30, 2016

Related U.S. Application Data
(60) Provisional application No. 62/236,162, filed on Oct. 2, 2015.

(51) Int. Cl. A61M 11/04; A61M 15/06; H05B 1/02; A61M 15/00

(52) U.S. Cl. CPC A61M 11/042; A61M 15/0021; A61M 15/06; H05B 1/0244; A61M 2205/8206; A61M 2205/3368; A61M 2205/3606; H05B 2203/021

(57) ABSTRACT

A novel Aromatherapy Vaporization Device is disclosed having a heating chamber first portion with an airflow restricting member and a vapor capturing chamber disposed upstream thereof. A heating chamber second portion has a low thermal inertia conductive heating element having a control circuit first electrical coupling. Phyto material is inserted between the first and second heating chamber portions for heating thereof to a predetermined temperature. A hinge is disposed between the heating chamber first portion and the heating chamber second portion for facilitating loading of phyto material into the heating chamber.

### CROSS REFERENCE TO RELATED APPLICATION

This application benefits from the priority of U.S. Provisional Applications 62/236,162 filed on Oct. 2, 2015, which is incorporated herein by reference.

### TECHNICAL FIELD OF THE INVENTION

The technical field relates to a device for vaporization of phyto materials and more specifically to a device for use in aromatherapy vaporization.

### BACKGROUND OF THE INVENTION

Aromatherapy generally uses essential oils, which are extracted from phyto materials, such as leaves of plants, for therapeutic benefits. These essential oils are either massaged into the skin or can be inhaled. In some cases the phyto materials are heated in order to released the essential oils therefrom. By heating these phyto materials at predetermined temperatures, essential oils and extracts are boiled off, depending upon the temperature at which these phyto materials are heated, a vapor, which is a faintly visible suspension of fine particles of matter in the air or aerosol, which is a gaseous suspension of fine solid or liquid particles, is given off, which is then inhaled by a user for its therapeutic benefits.

Devices that provide such operation are generally known as vaporizers and they generally fall into two categories. These are convection and conduction. Convection vaporizers pass hot air at a predetermined temperature through the ground leaf materials to extract the various essential oils to generate the vapor, whereas conduction vaporizers provide heat to the phyto materials through direct contact between the phyto materials and a heating chamber to generate the vapor.

Different phyto materials release vapor at different temperatures. Some release vapor at 120 degrees Celsius, whereas others at 220 degrees Celsius. The predetermined temperature is less than a combustion temperature of the phyto material. In some of the prior art vaporization devices the vapor released from the materials are quite hot, around 230 degrees Celsius and may cause discomfort when inhaled by a user, which is typically a result of the close proximity of the users lips to heating source used for vaporizing of the phyto material. In many prior art vaporizer devices, the heating chamber is very close to the lips of the user, in some cases less than 2 centimeters away. This means that the hot air and vapor mixture may easily cause discomfort as well as potentially to burn the lips of the user. In addition, many prior art vaporization devices allow for no restricted airflow and the user can inhale a lot of air and not a lot of vapors that are released from the phyto material. The inhalation of a lot of hot air as well as the hot air not having much phyto material vapors contained therein is not advantageous.

It is therefore an object of the invention to provide an aromatherapy vaporization device that overcomes the aforementioned deficiencies.

### SUMMARY OF THE INVENTION

In accordance with the embodiments of the invention there is provided: a herbal vaporization device comprising: a first housing comprising a first end and a second end upstream of the first end and a fluid pathway starting from a downstream ambient air input port disposed proximate the first end and propagating to the second end and terminating at an inhalation aperture proximate the second end, the fluid pathway comprising: a heating chamber fluidly coupled with the downstream ambient air input port in fluid communication with an outside environment through the ambient air input port and fluidly coupled with an upstream vapor and air cooling member, wherein the inhalation aperture is disposed upstream of the vapor and air cooling member, the heating chamber for in a first mode of operation for receiving of phyto material therein and for in a second mode of operation for heating of the phyto material at a predetermined temperature and for heating of the phyto material for at least partial vaporization thereof, the heating chamber comprising: a heating chamber first portion and a heating chamber second portion, wherein the heating chamber first and second portions in the second mode of operation have the first portion and the second portion proximate each other and form an approximately enclosed fluid pathway and where in the first mode of operation form an other than approximately enclosed fluid pathway and allow for the receiving of phyto material therein; the heating chamber first portion comprising an airflow restricting member and a vapor capturing chamber disposed upstream thereof; the heating chamber second portion comprising a low thermal inertia conductive heating element having a control circuit first electrical coupling; a first battery at least partially disposed within the first housing; a first control circuit coupled with the first battery and the control circuit first electrical coupling, the first control circuit for controlling the flow of electrical current from the first battery to the low thermal inertia conductive heating element; a first battery first recharging port coupled with the first control circuit; a switch coupled with the first control circuit for providing a control signal to the first control circuit for affecting the flow of electrical current from the first battery to the low thermal inertia conductive heating element, wherein in use, in the second mode of operation upon activating the switch, electrical current is applied to the control circuit first electrical coupling and the low thermal inertia conductive heating element is heated to the predetermined temperature and the phyto material is heated and vapor emitted therefrom propagates through apertures of a predetermined size formed in the airflow restricting member and into the vapor capturing chamber for further propagation into the vapor and air cooling member for inhalation from the inhalation aperture, wherein further in use, the phyto material in the second mode of operation is compressed between the airflow restricting member and the low thermal inertia conductive heating element.

In accordance with the embodiments of the invention there is provided a herbal vaporization device comprising: a first housing comprising a first end and a second end upstream of the first end and a fluid pathway starting from a downstream ambient air input port disposed proximate the first end and propagating to the second end and terminating at an inhalation aperture proximate the second end, the fluid pathway comprising: a heating chamber fluidly coupled with the downstream ambient air input port in fluid communication with an outside environment through the ambient air input port and fluidly coupled with an upstream vapor and air cooling member, wherein the inhalation aperture is disposed upstream of the vapor and air cooling member, the heating chamber for in a first mode of operation for receiving of phyto material therein and for in a second mode of operation for heating of the phyto material at a predetermined temperature and for heating of the phyto material for at least partial vaporization thereof, the heating chamber comprising: a heating chamber first portion and a heating chamber second portion, wherein the heating chamber first and second portions in the second mode of operation have the first portion and the second portion proximate each other and form an approximately enclosed fluid pathway and where in the first mode of operation form an other than approximately enclosed fluid pathway and allow for the receiving of phyto material therein, the heating chamber first portion comprising an airflow restricting member and a vapor capturing chamber disposed upstream thereof, the heating chamber second portion comprising a low thermal inertia conductive heating element having a control circuit first electrical coupling; a hinge disposed between the heating chamber first portion and the heating chamber second portion for allowing the operation of the heating chamber between the first mode and the second mode of operation; a thermal insulating layer comprising an air cavity disposed proximate the low thermal inertia conductive heating element on an opposite side where the low thermal inertia conductive heating element is for contacting the phyto material; a first battery at least partially disposed within the first housing; a first control circuit coupled with the first battery and the control circuit first electrical coupling, the first control circuit for controlling the flow of electrical current from the first battery to the low thermal inertia conductive heating element; a first battery first recharging port coupled with the first control circuit; a switch coupled with the first control circuit for providing a control signal to the first control circuit for affecting the flow of electrical current from the first battery to the low thermal inertia conductive heating element, wherein in use, in the second mode of operation upon activating the switch, electrical current is applied to the control circuit first electrical coupling and the low thermal inertia conductive heating element is heated to the predetermined temperature and the phyto material is heated and vapor emitted therefrom propagates through apertures of a predetermined size formed in the airflow restricting member and into the vapor capturing chamber for further propagation into the vapor and air cooling member for inhalation from the inhalation aperture, wherein further in use, the phyto material in the second mode of operation is compressed between the airflow restricting member and the low thermal inertia conductive heating element.

### BRIEF DESCRIPTION OF THE DRAWINGS

FIG. 1A illustrates a herbal vaporization device in accordance with a first embodiment of the invention and in a first mode of operation;

FIG. 1B illustrates a herbal vaporization device in accordance with a first embodiment of the invention and in a second mode of operation;

FIG. 1C illustrates a cutaway view of the herbal vaporization device with a focus on the heating chamber; and

FIG. 1D illustrates the herbal vaporization device with a housing removed.

### DETAILED DESCRIPTION OF EMBODIMENTS OF THE INVENTION

For the purposes of this detailed description, the term loose leaf herbal material is categorized as phyto material 419 and includes phyto material extract, where the phyto material extract is derived from the phyto material 419 or from the loose leaf herbal material.

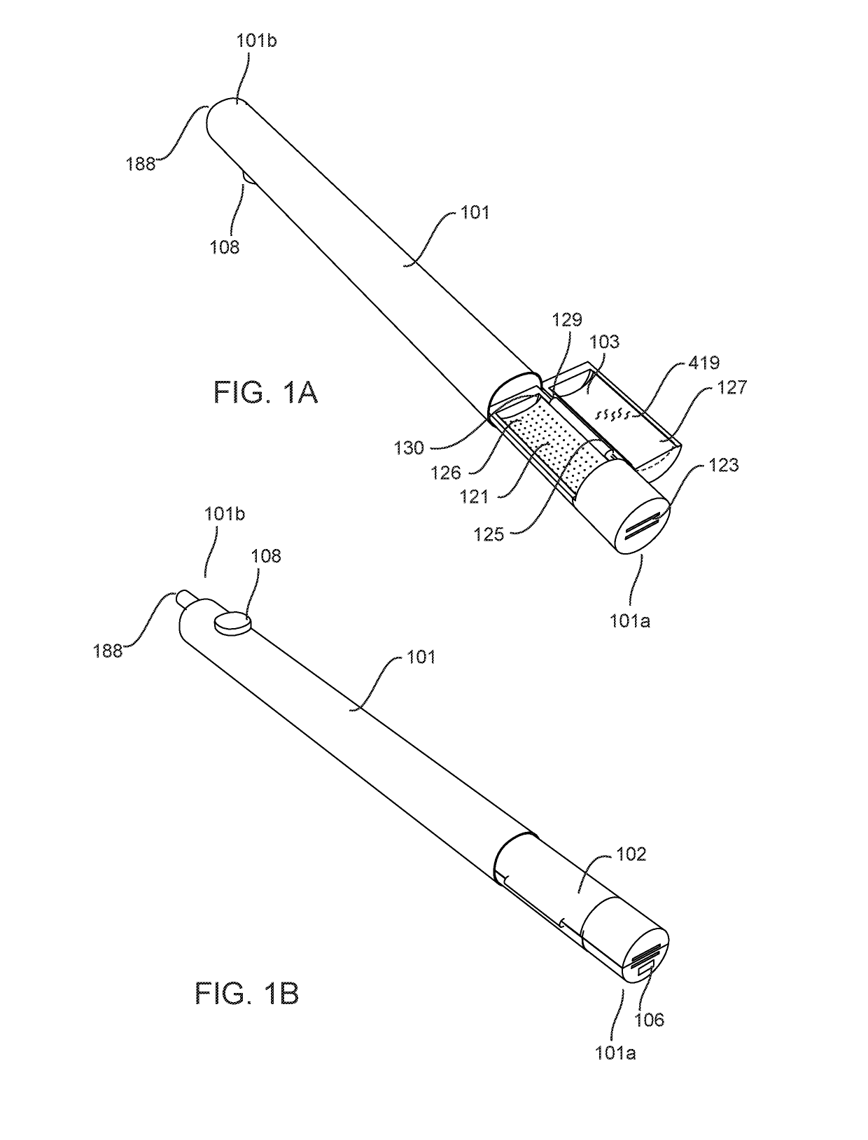

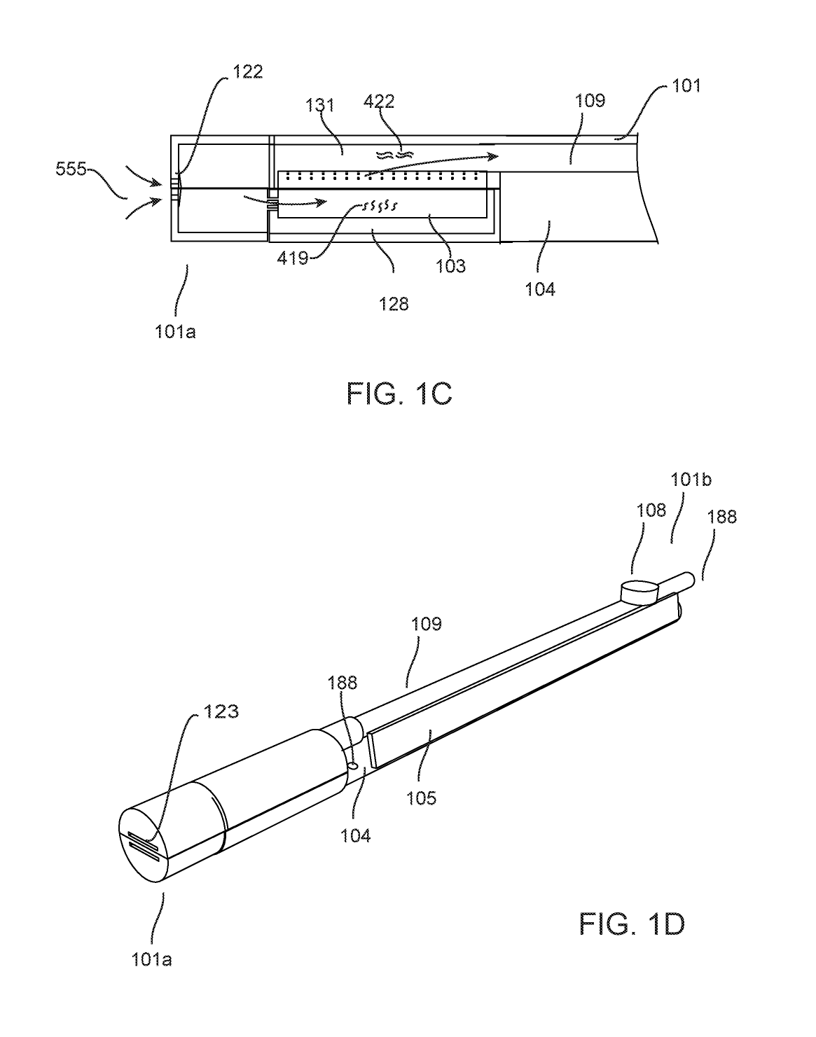

FIG. 1A illustrates a herbal vaporization device (HVD) 100 in accordance with a first embodiment of the invention. The HVD is formed from a first housing 101 having a first end 101a and a second end 101b upstream of the first end 101a and a fluid pathway 109 starting from a downstream ambient air input port 123 disposed proximate the first end and propagating to the second end and terminating at an inhalation aperture 188 proximate the second end 101b.

The fluid pathway 109 includes a heating chamber 102 fluidly coupled with the downstream ambient air input port 123 in fluid communication with an outside environment through the ambient air input port 123 and fluidly coupled with an upstream vapor and air cooling member 109, wherein the inhalation aperture 188 is disposed upstream of the vapor and air cooling member 109. Preferably the vapor and air cooling member 109 is manufactured from a thermally conductive material, such as copper or aluminum or in a last case stainless steel, to cool the vapor 422 (FIG. 1C) and air propagating therethrough to a temperature that is comfortable for inhalation by a user. Ideally a temperature that is comfortable for inhalation by a user is less than 40 degrees Celcius.

As is shown in FIG. 1A, the heating chamber 102 is shown in a first mode of operation for receiving of phyto material 419 therein. The heating chamber 102 includes a heating chamber first portion 126 and a heating chamber second portion 127 and in the first mode of operation form an other than approximately enclosed fluid pathway and allow for the receiving of phyto material 419 therein. Preferably the phyto material 419 is finely ground before being inserted into the heating chamber 102.

A hinge 125 is disposed between the heating chamber first portion 126 and the heating chamber second portion 127 for allowing the operation of the heating chamber between the first mode and the second mode of operation.

FIG. 1B illustrates the heating chamber 102 in a second mode of operation for heating of the phyo material 419 at a predetermined temperature and for heating of the phyto material 419 for at least partial vaporization thereof. The predetermined temperature is normally in the range of 160 degrees Celsius to 230 degrees Celsius. Ideally below the combustion temperature of the phyto material 419.

The heating chamber first and second portions, 126 and 127, in the second mode of operation have the first portion and the second portion proximate each other and form an approximately enclosed fluid pathway.

Referring to the heating chamber first portion 126, it includes an airflow restricting member 121 and a vapor capturing chamber 131 disposed upstream thereof. Having an airflow restricting member 121 allows for controlling a flow of air and vapor mixture propagating through the HVD 100 and as such provides for improved vapor production.

Referring to FIG. 1D in conjunction with FIG. 1A, the heating chamber second portion 127 comprising a low thermal inertia conductive heating element 103 and includes a control circuit first electrical coupling 129. Having a low thermal inertia conductive heating element 103 allows for faster heating.

In addition, a control circuit second electrical coupling 130 is electrically coupled with the airflow restricting member 121 and the first control circuit 105, wherein the airflow restricting member comprises a resistive metal material for heating to the predetermined temperature.

As is illustrated in FIG. 1D, with the HVD 100 having the housing 101 removed, the first battery 104 is at least partially disposed within the first housing 101 and a first control circuit 105 is coupled with the first battery 104 and the control circuit first electrical coupling 129.

The first control circuit 105 is for controlling the flow of electrical current from the first battery 104 to the low thermal inertia conductive heating element 103. Referring to FIG. 1B, a first battery first recharging port 106 is electrically coupled with the first control circuit 105. A switch 108 is electrically coupled with the first control circuit 105 for providing a control signal to the first control circuit 105 for affecting the flow of electrical current from the first battery 104 to the low thermal inertia conductive heating element 103.

Referring to FIG. 1C, a cutaway view of the HVD 100 is shown. In use of the HVD 100 in the second mode of operation, upon activating the switch 108, electrical current is applied to the control circuit first electrical coupling 129 and the low thermal inertia conductive heating element 103 is heated to the predetermined temperature and the phyto material 419 is heated and vapor 422 emitted therefrom propagates through apertures of a predetermined size (FIG. 1A) formed in the airflow restricting member 121 and into the vapor capturing chamber 131 for further propagation into the vapor and air cooling member 109 for inhalation from the inhalation aperture 188. For example the apertures of the predetermined size that are envisaged have a diameter of about 0.1 mm to 0.5 mm. A thermal insulating layer 128 is formed having an air cavity disposed proximate the low thermal inertia conductive heating element 103 on an opposite side where the low thermal inertia conductive heating element 103 is for contacting the phyto material 419.

Referring to FIG. 1D, optionally, a temperature sensor 188 is provided and electrically coupled with the first control circuit 105 for sensing a temperature of the heating chamber and for providing a temperature signal thereto in order to affect the predetermined temperature. So for example the predetermined temperature is less than a combustion temperature of the phyto material.

Referring to FIG. 1D, optionally, a temperature sensor 188 is provided and electrically coupled with the first control circuit 105 for sensing a temperature of the heating chamber and for providing a temperature signal thereto in order to affect the predetermined temperature. So for example upon pressing of the switch 108, the temperature signal is also used for affecting the flow of electrical current from the first battery 104 to the low thermal inertia conductive heating element 103. In such a manner the predetermined temperature will be maintained when the switch 108 is depressed and will not be exceeded by the first control circuit 105.

Advantageously, the phyto material 419 in the second mode of operation is compressed between the airflow restricting member 121 and the low thermal inertia conductive heating element 103. Compressing of the phyto material 419 generally provides for an improved vaporization thereof because compressing the phyto material 419 works better with conduction heating. Of course phyto material extract is also useable for being placed within the airflow restricting member 121 and the low thermal inertia conductive heating element 103. This will of course require additional cleaning of the heating chamber 102 after the vaporization thereof.

Advantageously, the first embodiment of the invention provide for a vaporization device that is fast heating because of the low thermal inertia of the conductive heating element 103. Furthermore, there is a large surface area provided for contacting of the phyto material 419 within the heating chamber between the airflow restricting member 121 and the low thermal inertia of the conductive heating element 103. This provides for a double advantage in the heating of the phyto material 419. Phyto material 419 that is spread on a large surface area and with little depth or height will provide for a more even vaporization in this case of conduction heating. Such an arrangement therefore potentially reduces the need to have to stir the material between vaporization sessions as it will be more evenly vaporized when used in the HVD 100 in accordance with the first embodiment of the invention.

Furthermore, when the heating chamber is opened up in the first mode of operation for loading of the phyto material 419 its easy to clean. A finger can be used to scoop out the vaporized phyto material and new phyto material is easily insertable.

Because of the low thermal inertia of the conductive heating element 103 it also has low power consumption as this is manufactured from a nickel alloy having a total resistance of about 0.25 Ohm to 2 Ohm.

Having a thermal insulating layer of air opposite the side where the low thermal inertia of the conductive heating element contacts the phyto material 419 allows for the low thermal inertia of the conductive heating element 103 to heat quickly and to be insulated from losing heat other than at its edges where it contacts the heating chamber second portion 127.

The single direction airflow valve 122 allows for ambient air to enter the heating chamber 102 and for vapor 422 to other than escape from the heating chamber 102 through this valve 122. An example of such a valve is an umbrella valve and preferably this valve 122 is manufactured from a silicone material.

Preferably the phyto material 419 is ground finely before being inserted into the heating chamber 102 for allowing of air to pass through the material from the ambient air input port 123 to the vapor capturing chamber 131. The single direction airflow valve 1222 also preferably results in a smell proof vaporization device during heating as vapor 422 does not escape through this valve.

Furthermore, when the heating chamber first portion 126 and the heating chamber second portion 127 are coupled together in the second mode of operation, there is a seal formed between the two portions that doesn't allow for vapor 422 to escape and for air to enter the heating chamber other than through the single direction airflow valve 122.

Having such a configuration for the HVD 100 advantageously makes for a small device that can then be easily adapted to fit into a pocket and is discreet when utilized.

Numerous other embodiments are envisaged without departing from the spirit or scope of the invention.

What I claim is:

1. A herbal vaporization device comprising:
a first housing comprising a first end and a second end upstream of the first end and a fluid pathway starting from a downstream ambient air input port disposed proximate the first end and propagating to the second end and terminating at an inhalation aperture proximate the second end, the fluid pathway comprising:
a heating chamber fluidly coupled with the downstream ambient air input port in fluid communication with an outside environment through the ambient air input port and fluidly coupled with an upstream vapor and air cooling member, wherein the inhalation aperture is disposed upstream of the vapor and air cooling member, the heating chamber for in a first mode of operation for receiving of phyto material therein and for in a second mode of operation for heating of the phyto material at a predetermined temperature and for heating of the phyto material for at least partial vaporization thereof, the heating chamber comprising:
a heating chamber first portion and a heating chamber second portion, wherein the heating chamber first and second portions in the second mode of operation have the first portion and the second portion proximate each other and form an approximately enclosed fluid pathway and where in the first mode of operation form an other than approximately enclosed fluid pathway and allow for the receiving of phyto material therein;
the heating chamber first portion comprising an airflow restricting member and a vapor capturing chamber disposed upstream thereof;
the heating chamber second portion comprising a low thermal inertia conductive heating element having a control circuit first electrical coupling;
a first battery at least partially disposed within the first housing;
a first control circuit coupled with the first battery and the control circuit first electrical coupling, the first control circuit for controlling the flow of electrical current from the first battery to the low thermal inertia conductive heating element;
a first battery first recharging port coupled with the first control circuit;
a switch coupled with the first control circuit for providing a control signal to the first control circuit for affecting the flow of electrical current from the first battery to the low thermal inertia conductive heating element,
wherein in use, in the second mode of operation upon activating the switch, electrical current is applied to the control circuit first electrical coupling and the low thermal inertia conductive heating element is heated to the predetermined temperature and the phyto material is heated and vapor emitted therefrom propagates through apertures of a predetermined size formed in the airflow restricting member and into the vapor capturing chamber for further propagation into the vapor and air cooling member for inhalation from the inhalation aperture,
wherein further in use, the phyto material in the second mode of operation is compressed between the airflow restricting member and the low thermal inertia conductive heating element.

2. A herbal vaporization device according to claim 1 comprising:
a control circuit second electrical coupling electrically coupled with the airflow restricting member and the first control circuit, wherein the airflow restricting member comprises a resistive metal material for heating to the predetermined temperature.

3. A herbal vaporization device according to claim 1 comprising:
a hinge disposed between the heating chamber first portion and the heating chamber second portion for allowing the operation of the heating chamber between the first mode and the second mode of operation.

4. A herbal vaporization device according to claim 1 wherein the vapor and air cooling member comprises a thermally conductive material to cool the vapor and air propagating therethrough to a temperature that is comfortable for inhalation by a user.

5. A herbal vaporization device according to claim 1 comprising:
a single direction airflow valve disposed upstream of downstream ambient air input port for allowing of ambient air to propagate into the heating chamber in an upstream direction only.

6. A herbal vaporization device according to claim 1 comprising:
a thermal insulating layer comprising an air cavity disposed proximate the low thermal inertia conductive heating element on an opposite side where the low thermal inertia conductive heating element is for contacting the phyto material.

7. A herbal vaporization device according to claim 5 wherein the single direction airflow valve comprises an umbrella valve.

8. A herbal vaporization device according to claim 1 wherein the low thermal inertia conductive heating element comprises a metal material and comprises a nickel metal alloy.

9. A herbal vaporization device according to claim 1, comprising temperature sensor is provided and electrically coupled with the first control circuit for sensing a temperature of the heating chamber and for providing a temperature signal thereto in order to affect the predetermined temperature.

10. A herbal vaporization device comprising:
a first housing comprising a first end and a second end upstream of the first end and a fluid pathway starting from a downstream ambient air input port disposed proximate the first end and propagating to the second end and terminating at an inhalation aperture proximate the second end, the fluid pathway comprising:
a heating chamber fluidly coupled with the downstream ambient air input port in fluid communication with an outside environment through the ambient air input port and fluidly coupled with an upstream vapor and air cooling member, wherein the inhalation aperture is disposed upstream of the vapor and air cooling member, the heating chamber for in a first mode of operation for receiving of phyto material therein and for in a second mode of operation for heating of the phyto material at a predetermined temperature and for heating of the phyto material for at least partial vaporization thereof, the heating chamber comprising:
a heating chamber first portion and a heating chamber second portion, wherein the heating chamber first and second portions in the second mode of operation have the first portion and the second portion proximate each other and form an approximately enclosed fluid pathway and where in the first mode of operation form an other than approximately enclosed fluid pathway and allow for the receiving of phyto material therein,
the heating chamber first portion comprising an airflow restricting member and a vapor capturing chamber disposed upstream thereof,
the heating chamber second portion comprising a low thermal inertia conductive heating element having a control circuit first electrical coupling;
a hinge disposed between the heating chamber first portion and the healing chamber second portion for allowing the operation of the heating chamber between the first mode and the second mode of operation;
a thermal insulating layer comprising an air cavity disposed proximate the low thermal inertia conductive heating element on an opposite side where the low thermal inertia conductive heating element is for contacting the phyto material;
a first battery at least partially disposed within the first housing;
a first control circuit coupled with the first battery and the control circuit first electrical coupling, the first control circuit for controlling the flow of electrical current from the first battery to the low thermal inertia conductive heating element;
a first battery first recharging port coupled With the first control circuit;
a switch coupled with the first control circuit for providing a control signal to the first control circuit for affecting the flow of electrical current from the first battery to the low thermal inertia conductive heating element,
wherein in use, in the second mode of operation upon activating the switch, electrical current is applied to the control circuit first electrical coupling and the low thermal inertia conductive heating element is heated to the predetermined temperature and the phyto material is heated and vapor emitted therefrom propagates through apertures of a predetermined size formed in the airflow restricting member and into the vapor capturing chamber for further propagation into the vapor and air cooling member for inhalation from the inhalation aperture,
wherein further in use, the phyto material in the second mode of operation is compressed between the airflow restricting member and the low thermal inertia conductive heating element.
